(19) United States
(12) Patent Application Publication
(10) Pub. No.: US 2008/0103403 A1
(43) Pub. Date: May 1, 2008

(54) Method and System for Diagnosis of Cardiac Diseases Utilizing Neural Networks

(76) Inventor: Eyal Cohen, Tel Aviv (IL)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/718,840

(22) PCT Filed: Nov. 7, 2005
(86) PCT No.: PCT/IL05/01162

§ 371 Date: Sep. 12, 2007

(30) Foreign Application Priority Data
Nov. 8, 2004 (IL) 165096

(51) Int. Cl. A61B 5/0402

(52) U.S. Cl. 600/509; 706/20

(57) ABSTRACT

The present invention is directed to a method for diagnosing silent and/or symptomatic cardiac diseases in human patients, based on extracting and analyzing hidden factors or a combination of hidden and known factors of ECG signals. The diagnosis method employs rest-ECG signals of a group of diagnosed patients, the group consisting of patients a-priori diagnosed as sick patients and of patients a-priori diagnosed as healthy patients by trusted procedures. Artificial neural networks are then iteratively trained to accurately classify the cardiac disease by processing the corresponding raw input signals of the diagnosed patients. The weights and biases data representing the trained neural networks are saved. Unknown, new patients are diagnosed as sick or healthy patients by processing their corresponding raw ECG signals by the trained neural networks.

### FIELD OF THE INVENTION

The present invention relates to the field of medical signals analysis based on Machine Learning processes. More particularly, the invention relates to a method and system for diagnosing cardiac diseases, based on factors obtained by employing Artificial Neural Network processing of medical signals.

### BACKGROUND OF THE INVENTION

Ischemia is an insufficient supply of blood to an organ, usually due to a blocked artery. Myocardial ischemia is an intermediate condition in coronary artery disease during which the heart tissue is slowly or suddenly starved of oxygen and other nutrients. Eventually, when blood flow to the heart is completely blocked, the affected heart tissue will die leading to a heart attack. Yet, only 15% of heart attacks happen this way. Pathologists have demonstrated that most attacks occur after a plaque fibrous cap on the artery internal wall breaks open, promoting a blood clot to develop over the break. The clot blocks the artery, and a heart attack is inevitable and sudden (Libby. P., Atherosclerosis: The new view. Scientific American, May 2002, 29-37.). Ischemia can be symptomatic (physical and diagnostical) or silent (i.e., without symptoms). According to the American Heart Association, up to four million Americans may have silent ischemia and be at high risk of having a heart attack with no early warning.

Diagnostic tests for myocardial ischemia include: rest, exercise, or ambulatory ElectroCardioGrams (ECGs); scintigraphic studies (radioactive heart scans); echocardiography; coronary angiography; and, rarely, positron emission tomography. However, the most reliable diagnosis of the cardiac arteries condition is the catheterization procedure. Notably, except for the rest-ECG, these tests are expensive, less accessible, and in the case of catheterization, also invasive and carry risk to the patient.

An ECG shows the heart's electrical activity and may reveal a lack of oxygen supply to the heart muscles. Impulses of the heart's activity are recorded by the ECG monitoring devices on paper, or digitally. The standard, rest-ECG test takes about 10 minutes and it is performed in a physician's office. Another type of electrocardiogram, known as the exercise stress test, measures the response to exertion when the patient is exercising on a treadmill or a stationary bike. It is performed in a physician's office or an exercise laboratory and takes 15 to 30 minutes. This test is more reliable than a resting ECG in diagnosing ischemia. Sometimes an ambulatory ECG is ordered, wherein the patient wears a portable ECG monitoring machine, called a Holter monitor, for 12, 24, or 48 hours.

Diagnosis of cardiac diseases, based on ECG recordings, usually employs rule-based criteria, namely: measuring and analyzing well defined “intervals”, “segments” and “waves” of the heart impulse signal (FIG. 1). In many cases the diagnosis may rely on a visual inspection by an expert cardiologist, capable of analyzing the plot morphology. For Example, FIGS. 2A and 2B demonstrate changes in ECG morphology that may indicate ischemia. FIG. 2A shows a normal heart impulse signal, and FIG. 2B shows a heart impulse signal with ST (ST Segment, FIG. 1) changes (i.e., with a deviated ST Segment), in which an apparent reversal of the T-wave (FIG. 1) is seen at the end of the heart cycle—a possible indication of ischemia.

However, such ‘rule-based’ diagnosis criteria are inefficient and inaccurate. Many rest-ECGs of cardiac disease patients, who did not suffer from a heart attack, seem normal under visual inspection. In fact, about 25% of patients with angina pectoris (i.e., suffer from physical symptoms as chest pain, tightness or heaviness in the chest) have normal ECGs. Moreover, some of these patients with physical complaints may not suffer from ischemia at all. This does not mean that rest-ECGs do not carry any reliable information about the cardiac disease. In fact, feasibility tests, employing neural networks have demonstrated that rest-ECGs carry salient information (in the form of hidden factors) about the condition of the cardiac system. These factors may be complex, and thus invisible even to an expert cardiologist's eye. However, they may be revealed using machine learning methods, such as artificial Neural Networks (NN) or Support Vector Machines (SVM). Such methods produce these hidden factors “internally” by scanning a database of pre-diagnosed ECGs, without the need for further a-priori knowledge.

Several patents disclose methods for processing medical signals that employ NN for ECG analysis. These patents analyze common ECG factors, e.g., the QRS complex (U.S. Pat. Nos. 5,020,540 and 5,947,909); or, they analyze data that was extracted from ECG signals by other than NN means (WO 01/82099 A1); or, they do not diagnose Cardiac Diseases (U.S. Pat. No. 5,640,966 and EP 0712605A1 detect ECG electrodes which are erroneously attached to the patient); or, they detect Cardiac Arrhythmia or Ventricular Tachycardia, which produce a significantly different and easily detected signals (U.S. Pat. Nos. 5,280,792 5,251,626 6,192,273 and 5,280,792). However, none of these patents employ NN based, pattern recognition processes, to diagnose Cardiac Diseases based on unknown, hidden factors. Furthermore, none of these patents is capable of diagnosing Cardiac Diseases in normal (i.e., healthy) looking rest-ECG.

There is therefore an ongoing need to provide inexpensive and non-invasive means for carrying out Diagnosis by Hidden Factors (DHF) for (early) diagnosis of cardiac diseases.

The present invention aims at providing a method and system for diagnosing cardiac diseases, based on standard, rest-ECG recordings.

It is also an object of the present invention to provide a method and system for diagnosing cardiac diseases, based on ‘machine learning’ (i.e., learning from examples) classification processes.

It is also an object of the present invention to provide a method and system for carrying out DHF, based on pattern recognition and classification processes.

It is another object of the present invention to provide a method and system for carrying out DHF that produces its own hidden factors by training NNs according to a-priori diagnosed ECG examples.

It is a further object of the present invention to provide a DHF of cardiac diseases, based on pattern recognition and classification processes utilizing standard rest-ECG recordings.

It is a still another object of the present invention to provide neural networks architecture and dynamics for carrying out DHF.

It is an additional object of the present invention to provide a combination of methods for optimizing the generalization capability of DHF.

Other objects and advantages of the invention will become apparent as the description proceeds.

### SUMMARY OF THE INVENTION

The following terms are defined in order to better understand the invention:

ECG (ElectroCardioGram): a record of the electrical activity in the heart during the cardiac cycles.

ECG Leads: A scheme of electrode attachments to the body, linked via an electrical wire for measuring electrical signals from the heart. There are 12 standard leads:

Lead 1 (L_{I}): Connections to the two arms.

Lead 2 (L_{II}): Connections to the right arm and foot.

Lead 3 (L_{III}): Connections to the left arm and foot.

Lead 4 (aVR): Augmented-voltage connection to the right arm.

Lead 5 (aVL): Augmented-voltage connection to the left arm.

Lead 6 (aVF): Augmented-voltage connection to the foot.

Leads 7-12 (V1-V6): The six chest connections.

The following filters are standard in ECG recordings:

Notch filter: Removes a narrow slice of frequencies from the filtered signal. The notch filter removes from the ECG signal frequency harmonics that are induced by the domestic electricity network. Such that for a given electricity network operating with a network frequency of f Hz, the notch filter preferably removes from the ECG signal the f, 2f and 3f, harmonics (e.g., for f=50 Hz of the Israeli electricity network the said harmonics are 50, 100 and 150 Hz.

Baseline filter: A High-Pass filter which removes low frequencies of the breathing cycle.

EMG filter: Removes noise in a gradually increasing rejection magnitude, from 10 Hz and above.

The present invention is directed to a method for diagnosing silent and/or symptomatic cardiac diseases in human patients, based on extracting and analyzing hidden factors or a combination of hidden and known factors of ECG signals. The diagnosis method employs rest-ECG signals of a group of diagnosed patients that are acquired by any ECG recording unit. This group consists of patients, a-priori diagnosed as sick patients and of patients, a-priori diagnosed as healthy patients by trusted procedures. Furthermore, all signals of healthy and sick patients are diagnosed as healthy, according to standard, ‘rule-based’, visual methods of ECG diagnosis. Alternatively, all signals of healthy and sick patients are diagnosed as sick according to standard, ‘rule-based’ visual methods.

Artificial neural networks are then iteratively trained to accurately classify the cardiac disease by processing the corresponding raw (i.e., pre-processed but not analyzed rest-ECG) input signals of the diagnosed patients. Whenever required, training network cycles are added, until predetermined training performance conditions are satisfied. During the iterative training, diagnosed patients that have raw input data that deteriorates the convergence of the training process, in a large portion of the trained neural networks, are excluded from the group. The weights and biases data representing the trained neural networks are saved. Unknown, new patients are diagnosed as sick or healthy patients by processing their corresponding raw ECG signals by the trained neural networks.

More specifically, rest-ECG signals of patients a-priori diagnosed as sick patients, and of patients a-priori diagnosed as healthy patients by a trusted procedure such as catheterization, are acquired. Furthermore, all rest-ECG signals of both healthy and sick patients, are diagnosed as ‘healthy’ (alternatively, all rest-ECG signals of both healthy and sick patients, are diagnosed as ‘sick’) according to standard, ‘rule-based’ visual methods. These signals are first processed to obtain filtered input-signals, defined within a single heart cycle, aligned about the same isoelectric reference and normalized within predefined boundaries. Signals of sick and of healthy patients are randomly separated into ‘train’ and ‘test’ sets, where each set containing signals of both healthy and sick patients. A multilayer artificial neural network is iteratively trained to correctly classify the diagnosed patients, by forwarding the signals of the train-set through the network, comparing the network output with the trusted diagnosis, and updating weights and biases data of the network accordingly. Each time, inputs that correspond to the diagnosed patients are fed into the network, while providing weights and biases data to each cycle, and updating them using error minimization techniques, until a predetermined training performance condition is satisfied or deteriorated. The trained network is then tested by processing the inputs that correspond to the selected test-set signals and the test results of the trained network are maintained. Trained networks are added by repeating this process, until a predetermined test-performance condition, based on the aggregated test results of all trained networks is satisfied. Inputs that consistently contribute a significant error in the training process of the trained networks are disqualified and the training process is repeated with the reduced set of inputs, and for a number of ECG Lead signals. The final weights and biases data obtained by each of the trained neural networks are saved. Then, new ECG signals of unknown (i.e., that were not included in the training phase) patients are acquired and processed to obtain new filtered input-signals, aligned about the same isoelectric reference and normalized using exactly the same formula that was applied for processing the a-priori diagnosed signals. Each of the new signals is applied as an input of the trained neural networks, while utilizing the saved weights and biases data and transforming the output results of each new signal to obtain “sick” or “healthy” classifications by the networks. Then, each of the new signals is classified as sick or healthy according to the majority of the networks classification results obtained for each signal of each lead separately. Finally, each of the unknown patients is diagnosed according to the majority of Leads classifications of his signals, while considering the majority of results obtained from the various Leads.

Diagnosis of new patients (i.e., generalization) is improved by any combination of generalization-improvement techniques, such as optimizing the NN architecture and/or ‘regularization’ of the performance function and/or ‘early stopping’ of the training process.

Preferably, processing is performed by filtering each acquired signal with a High-Pass filter, a notch filter, an EMG filter, or any combination thereof, before extracting a raw-input signal from each of the filtered signals, wherein the raw-input signal comprises a segment within a single heart cycle. All raw-input signals are aligned about the same isoelectric reference and the aligned raw-input signals are normalized within predetermined upper and lower boundaries.

The single cycles extracted from each of the signals are of the same time interval, and taken starting at the same predefined time interval before the peak of an R-wave and may be about 600 milliseconds long. The predefined time interval may be about 80 milliseconds. The upper bound may be larger than 0.75 and smaller than 1 and the lower bound may be smaller than 0.25 and larger than 0. Prior to, or during the processing phase, the ECG signals are converted into digital format, preferably by utilizing a sampling frequency of about 500 HZ.

Training is carried out utilizing signals of healthy and sick patients which are all diagnosed as healthy, according to standard, rule-based, visual methods or alternatively, by utilizing signals of healthy and sick patients which are all diagnosed as sick patients according to standard, rule-based, visual methods.

The present invention is also directed to a system for diagnosing cardiac diseases in unknown patients, based on extracting and analyzing hidden factors or a combination of hidden and known factors of rest-ECG signals, that comprises:

a database of a-priori diagnosed ECG signals of sick and of healthy patients, where the patients are diagnosed via a trusted procedure;

a signal processing unit for digitizing and processing the signals and for iteratively training multilayer artificial neural networks to correctly classify the diagnosed patients, by processing their corresponding raw input data, while whenever required, adding trained network cycles, until predetermined training and testing performance conditions are satisfied;

a memory for saving the weights and biases data representing the trained neural networks; and

a classification module for diagnosing unknown patients as sick or healthy patients by processing their corresponding raw signals by the trained neural networks.

More specifically, the system comprises:

a database of a-priori diagnosed ECG signals of sick and of healthy patients, where the patients are diagnosed via a trusted procedure;

a signal processing unit for processing the signals to obtain input-signals aligned about the same isoelectric reference and normalized within predefined boundaries, and for training artificial neural networks utilizing weights and biases data obtained;

a memory for saving weights and biases data of artificial neural networks; and

a classification module for acquiring new ECG signals of a non-diagnosed patient and processing the new signals, to obtain new input-signals aligned about the same isoelectric reference and normalized within the same predefined boundaries used by the signal processing unit. The classification module comprises sets of trained artificial neural networks for diagnosing the new signals utilizing the weights and biases data stored in the memory.

The system may further comprise a training unit for training an artificial neural network, in which training is performed by randomly selecting signals of sick and healthy patients from the database of a-priori diagnosed ECG signals, to be used for training and for testing of the training, and in which training is continuously carried out with all the train and test signals in the database, until predetermined training and generalization performance conditions are satisfied.

The processing unit may include filters for removing interfering signals from the cardiac signal and processing means for extracting a raw-input signal from the filtered signals, wherein the raw-input signal comprises a segment within a single cycle, for aligning the raw-input signals about the same isoelectric reference, and for normalizing the aligned raw-input signals within predetermined upper and lower boundaries.

### BRIEF DESCRIPTION OF THE DRAWINGS

In the drawings:

FIG. 1 illustrates the morphology elements employed in “rule based” diagnosis of ECG: The “intervals”, “segments” and “waves” defined within a single heart cycle;

FIG. 2A-B demonstrates changes in ECG morphology that indicate a possible ischemia;

FIG. 3 is a block diagram demonstrating the NN Feed Forward architecture;

FIG. 4 graphically demonstrates raw-input segmentation of a Lead 1 ECG signal, digitized with a 500 Hz sampling rate;

FIGS. 5A to 5C illustrate a possible frequency response of the High-Pass, notch, and EMG filters employed in processing the ECG signal;

FIG. 6 demonstrates the alignment of the raw-input signals about a common isoelectric reference value, and their normalization within given boundaries;

FIG. 7 is a block diagram illustrating a preferred embodiment of the DHF system of the invention;

FIG. 8 is a flowchart showing the initial stages of generating the database for training the neural networks;

FIG. 9 is a flowchart of the preprocessing steps of the raw ECG signal according to a preferred embodiment of the invention;

FIG. 10 is a flowchart showing the steps of the first training cycle of the NN according to a preferred embodiment of the invention;

FIG. 11 is a flowchart illustrating a preferred process for determining the sets of classifying-networks for the DHF of the invention; and

FIG. 12 is a flow chart illustrating the process of classifying a new signal of a non-diagnosed patient.

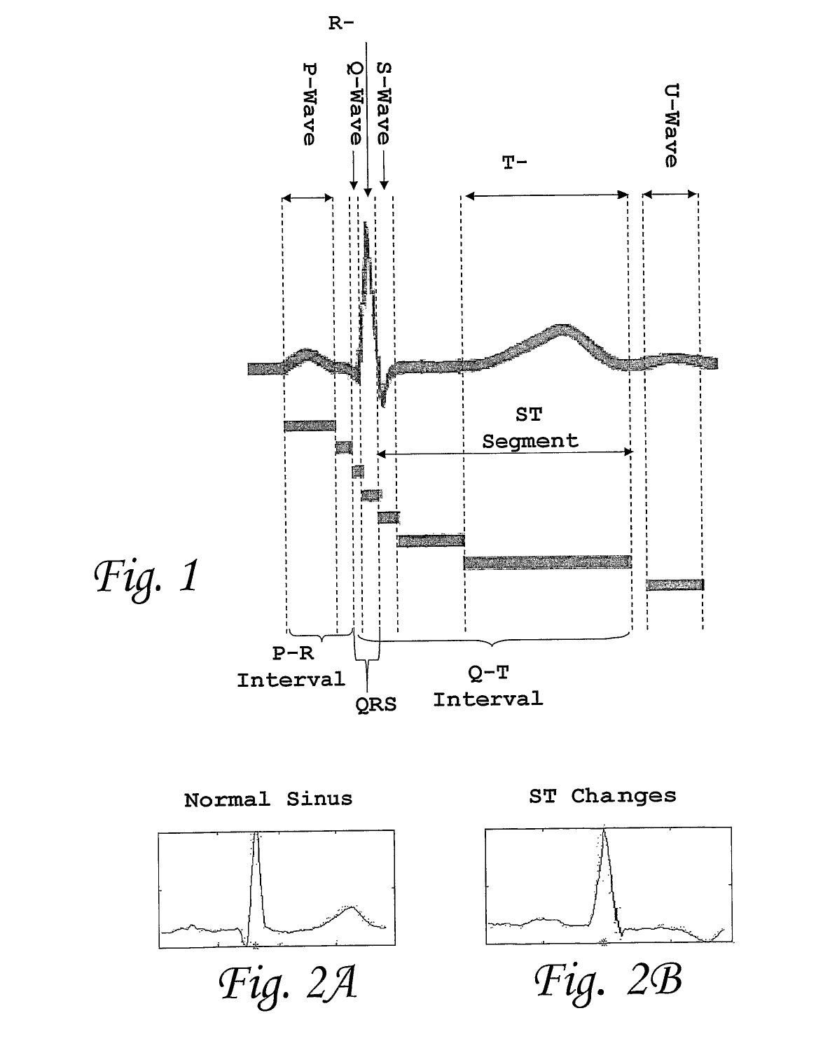

### DETAILED DESCRIPTION OF PREFERRED EMBODIMENTS

The Diagnosis by Hidden Factors (DHF) methods, disclosed by the present invention, extract hidden factors from ECG signals and track them, in order to produce a diagnosis of given cardiac diseases. The process is based on scanning a database of diagnosed a-priori (e.g., via catheterization) ECGs of healthy and sick patients, whose signals all look diagnostically alike to an expert cardiologist (i.e., either all patients' signals, healthy and sick, look healthy, or they all look sick).

The scan process is performed using sets of Neural Networks, which, being trained with the ECG examples, produce matrices of parameters, encoding the hidden factors of a given cardiac disease. The Neural Networks are capable of generalizing, namely, correctly diagnosing new ECGs that were not included in the scanned database.

The training and diagnosis of each cardiac disease are based on standard, rest-ECG recordings. Still, as feasibility tests demonstrated, DHF yields a significantly more reliable diagnosis compared with a diagnosis made by an expert cardiologist.

DHF is preferably performed as a parallel, distributed, trained by examples, pattern-recognition and classification task. Evidently, it is fundamentally different from the traditional rule-based, morphological methods which are currently employed by physicians and software.

### Neural Networks for Signal Classification

The classification task (e.g., classification of ECG signals of healthy and sick patients) solved by NNs can be defined as follows:

Given a database of N observations (herein after also referred to as “the training set”), where each observation is assigned a pair of vectors
A signal vector pⁿ (n=1, 2, . . . , N), comprising d elements (samples),

$${p}^{n} = [ \begin{matrix}{p}_{1}^{n} \\ {p}_{2}^{n} \\ ⋮ \\ {p}_{d}^{n}\end{matrix} ] ,$$

produced utilizing digital signal processing of a patient's heart impulse signal; and
an associated “truth” vector/value tⁿ given by a trusted source (e.g., preset according to a-priori trusted diagnosis of the patient).

In the case of the ECG classification task, pⁿ is a column vector of, for example, d=300 real values (originally voltage readings of the ECG signal), normalized in the range [0 1], 0<pᵢⁿ<1. The truth vector preferably has two possible states, wherein

$${t}^{n} = [ \begin{matrix}1 \\ 0\end{matrix} ]$$

indicates that the ECG was taken from a healthy patient, and

$${t}^{n} = [ \begin{matrix}0 \\ 1\end{matrix} ]$$

indicates that it was taken from a CD (with cardiac disease) patient. Alternatively, the dimensions of the truth vector may be reduced to tⁿ=0 for healthy, and tⁿ=1 for a CD patient. The “trusted diagnosis” is preferably constructed from a medical diagnosis based on catheterization or an equivalent procedure.

The NN task at the training phase is to find the correct classification for each input vector pⁿ, i.e., to perform the mapping pⁿ→tⁿ. To achieve this goal, the NN parameters are typically determined through a process of training, during which all observations {pⁿ, tⁿ} are iteratively processed by the NN while applying an error minimization algorithm. The training is stopped when the above mapping is performed correctly, or within a tolerable error, for all N observation pairs {pⁿ, tⁿ}.

Nevertheless, in order to become a practical classifier the NN is expected to generalize well. Namely, given a new input p^{new}, which it has not encountered in the process of training, the NN should yield the correct classification as “healthy” or “sick”. In practice, once completing the training of the NN, the correct classification of a new ECG is not given a-priori, but a well trained NN should yield the correct classification. This means that given a new patient's ECG signal (i.e., new processed heart impulse signal vector p^{new}), the well trained NN will classify it correctly as if the patient was diagnosed by catheterization.

An NN is defined by its architecture and dynamics.

### The “Feed Forward” (FF) Architecture:

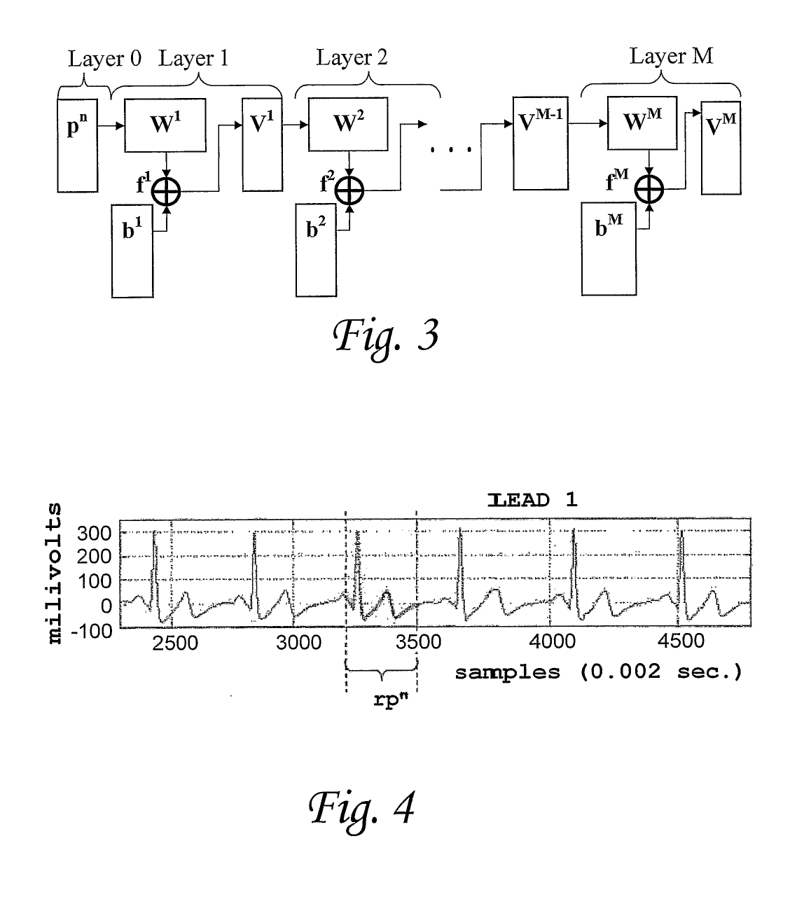

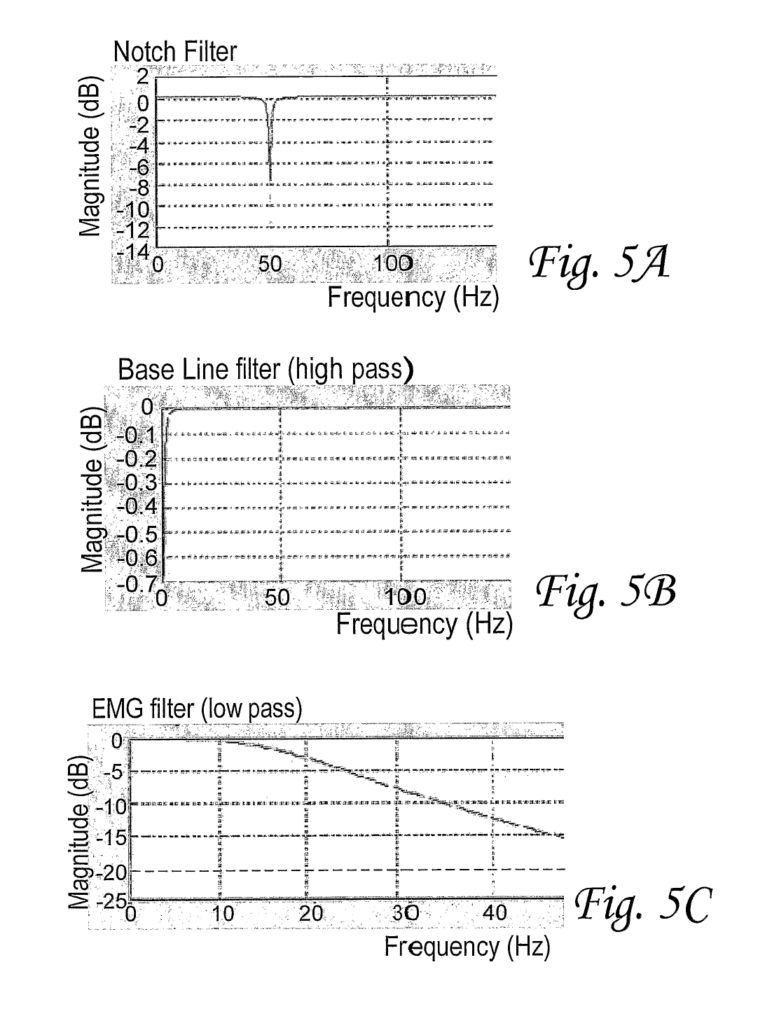

The FF architecture is arranged in M layers, as shown in FIG. 3. Each layer m (m=1, 2, . . . , M), except of the input layer (layer 0, which is the input vector pⁿ), consists of the following objects:

1. A weight matrix W^{m},

2. A bias vector b^{m}.

3. A “transfer function” ƒ^{m}. Common transfer functions used in NN implementations are:
ƒ(x)=sign(x) (“sign”);
ƒ(x)=α·x (“linear”);
ƒ(x)=(1+e^{-2·β·x})⁻¹ (“logistic”);

$$f ( x ) = \frac{{e}^{β x} - {e}^{- β x}}{{e}^{β x} + {e}^{- β x}}$$

(“hyperbolic tangens”)

4. All output vector V^{m}. ps Dynamics 1—Propagating a Signal:

As demonstrated in FIG. 3, the dynamics of propagating a signal through the feed forward NN architecture (Hertz, Krogh & Palmer: Introduction to the theory of neural computation. Addison-Wesley) are as follows:

1. Choose a signal vector pⁿ and process it through the 1^{st} layer, thereby producing the 1^{st} layer output vector: V¹=ƒ¹(W¹pⁿ+b¹), where η(h) indicates applying the transfer function ƒ on h; It should be noted that ƒ may differ from one layer to another.

2. Use the output vector of the 1^{st} layer, V¹, as input to the 2^{nd} layer, thereby producing the 2^{nd} layer output vector: V²=ƒ²(W²V¹+b²);

3. Repeat the process for all remaining layers (m=3, 4, . . . , M) to obtain the corresponding output vectors:

V^{m}=ƒ^{m}(h^{m})=ƒ^{m}(W^{m}V^{m-1}+b^{m})

where

h^{m}=W^{m}V^{m-1}+b^{m}

4. The resulting vector V^{M}, produced by the last layer, is the NN output Oⁿ (i.e., Oⁿ≡V^{M}) for the input of signal vector pⁿ. Note that during training Oⁿ may differ from the desired output tⁿ.

### Dynamics 2—Training the NN:

In a trained NN the weight matrices W^{m} and bias vectors b^{m} are adjusted to yield output vectors V^{M} which are close (within a tolerable error) to the associated “truth” value tⁿ: |Oⁿ−tⁿ|→0.

An NN may be trained to produce the expected outputs by applying the “(Error) Back Propagation” (BP) training algorithm, as follows:

1. Prepare the d×N inputs matrix P. Each column in P is a signal vector pⁿ (n=1, 2, . . . N) of length d;

2. Prepare a truth matrix T of length N. Each column tⁿ of T equals

$$[ \begin{matrix}1 \\ 0\end{matrix} ]$$

if the corresponding signal vector pⁿ is processed from the heart impulse signal (ECG) of a healthy patient, or

$$[ \begin{matrix}0 \\ 1\end{matrix} ]$$

whenever the ECG is of a sick (CD) patient;

3. Define the number of hidden layers M, and the size of each layer Sᵢₙₜ^{m}. It should be noted that the hidden layer size determines the size of the layer's output-vector: length(V^{m})=Sᵢₙₜ^{m}.

4. Choose an appropriate transfer function ƒ^{m} for each layer;

5. Initialize all weights and biases (W^{m} matrices and b^{m} vectors for all m values) to small random values. For example, if the size of the 1^{st} intermediate layer is Sᵢₙₜ¹, then the dimensions of the weight matrix W¹ of the 1^{st} layer will be Sᵢₙₜ¹×d, and it's bias vector b¹ will be Sᵢₙₜ¹×1, a Sᵢₙₜ¹-elements column vector.

6. Choose the first input vector signal p¹ (the first column of P) and propagate it forward through the network, as was described herein above, thereby producing the respective output vector of the NN: O¹=V^{M}. where O¹ is a 2-element vector comprising the elements O₁¹ and O₂¹.

7. Compute the weighted errors for the output layer: δᵢ^{M}=ƒ′(hᵢ^{M})·└tᵢ¹−Oᵢ¹┘, (where i=1,2; h^{m}=W^{m}V^{m-1}+b^{m}; hᵢ^{m} is the i-th element of h^{m} and therefore δᵢ^{M} is a 2×1 vector of the weights between the I neuron in the M−1 layer and the two output neuron of layer M; ƒ′ is the derivative of the transfer function, e.g., f′=2βf·(1−f) in the case of the logistic function).

8. Compute the weighted errors for the preceding layers by propagating the errors backwards: δᵢ^{m-1}=ƒ′(hᵢ^{m-1})·(W^{m}·δ^{m})ᵢ for m=M, M−1, . . . , 2 and i=1,2, . . . , Sᵢₙₜ^{m}.

9. Compute ΔW^{m}=η·δ^{m}·V^{m-1}, where η is the learning rate, preferably of order 0.01, and update all weights according to: W_{new}^{m}=W_{old}^{m}+ΔW^{m}.

10. Repeat steps 6-9 for the second input signal p².

11. Perform steps 6-9 for all N signal vectors in P. This completes one epoch (cycle) of training.

12. Train the network for a large number of epochs, i.e., repeat steps 6-11, until the mean squared classification error is smaller than a tolerable boundary value E_{T}:

$$Err = {\sum }_{i = 1}^{N} {[ {t}^{i} - {O}^{i} ]}^{2} < {E}_{T} ,$$

Where Err is termed also “the performance function”.

### Testing the NN Performance:

After the NN has reached the desired training performance (i.e., the output vectors of the training set corresponds with the expected truth values within the tolerable deviation error), freeze all elements of the weights W^{m} and biases b^{m}. To test the network generalization performance, a new signal vector p^{test} (i.e., one that was not used during training) is processed through the network utilizing the frozen weights and bias values, as follows:

1. V¹=ƒ¹(W¹·p^{test}+b¹);

2. V²=ƒ²(W²·V¹+b²)

3. Continue the process for all layers, thereby obtaining the output vectors:

V^{m}=ƒ^{m}(h^{m})=ƒ^{m}(W^{m}·V^{m-1}+b^{m}),
and yield the output vector of the last layer:

O^{test}≡V^{M}=ƒ^{M}(h^{M})=ƒ^{M}(W^{M}·V^{M-1}+b^{M})

4. Classify p^{test} according to the following decision rule, where

$${O}^{test} = [ \begin{matrix}{o}_{1} \\ {o}_{2}\end{matrix} ] \text{:}$$

If 0.5<o₁<1 and 0≦o₂≦0.5, then the ECG signal p^{test} is classified as “healthy”.
If 0≦o₁≦0.5 and 0.5<o₂≦1, than the ECG signal p^{test} is classified as “CD” (Cardiac Disease—“sick”).

### Improving Training Performance:

Adding Momentum

Performance can be improved during the training process by adding a momentum term in the computation of the weight changes (ΔW). More particularly, a momentum term is introduced into the weight change computation: ΔW^{m}(t)=η·δ^{m}·V^{m-1}+α·ΔW^{m}(t−1), where t refers to the current training cycle and t−1 to the preceding cycle. The momentum parameter α is set between 0 and 1, preferably about 0.9. Such an addition of momentum results in a faster training process that yields smaller Err (squared classification error) values.

Variable Learning Rate

The learning rate η can be adjusted to the progress of the training error Err, as follows:
If Err(t)>k·Err(t−1), where k>1, then η is decreased by a factor η_{dec} where η(t)=η_{dec}·η(t−1); and
If Err(t)<Err(t−1), then η is increased by a factor η_{inc}, where η(t)=η_{inc}·η(t−1).

The parameters k, η_{dec} and η_{inc}, are optimized by trial and error.

Other Training Processes:

The Back Propagation process described above is the most commonly used method for training NNs, but not necessarily the fastest. Other training processes exist that may result in a considerably shorter training runtimes. These processes include: Conjugate Gradient methods, in particular, the Scaled Conjugate Gradient Descent (see: Moller, M. F. A scaled conjugate gradient algorithm for fast supervised learning. Neural Networks 6(4): 525-533, 1993), Resilient Propagation (see: Riedmiller, M. and H. Braun. A direct adaptive method for faster backpropagation learning: The RPROP algorithm. IEEE International Conference on Neural Networks (San Francisco), vol. 1, pp. 586-591. IEEE, New York. 1993), The Lenenberg-Marquardt Method (see: Hagan, M. and M. Menhaj. Training feedforward networks with the Marquardt algorithm. IEEE Transactions on Neural Networks. 5(6): 989-993, 1994). These processes and others are available commercially (see: matlab—Neural Networks Toolbox Manual). The choice of the optimal training process is based on running benchmark tests of the different processes, using the same input and output matrices P, T, and comparing runtimes and generalization performance.

### Improving Generalization

Training a NN to the smallest possible error ET may result in overfitting. Namely—the NN performs well when tested with the trained data, but fails to classify new signals (i.e., poor generalization). Since the NN generalization performance is crucial for the applicability of the invention, it may be improved by utilizing one or more of the following methods:

Optimizing network architecture: the larger the network, i.e., with more and larger intermediate layers, the more it is adjustable to a specific database (overfitting) and as a result it cannot generalize well. Therefore the network architecture should be the slimmest possible, i.e., with the minimal number, of the smallest hidden layers. The exact architecture is preferably determined by ‘Cross-Validation’ and ‘Bootstrap’ methods, or by trial and error (see: Model selection with cross-validations and bootstraps—by A. Landasse, V. Wertz & M. Verleysen; ICNN/ICONIP 2003, LNCS 2714, pp. 573-580).

Regularization: the performance function,

$$Err = {\sum }_{i = 1}^{N} {( {T}^{i} - {O}^{i} )}^{2} < {E}_{T} ,$$

may be modified by adding a term that accounts for all the network weights and biases: RegErr=γ·Err+(1−γ)·[Σw_{i,j}²+Σbⱼ²], where w_{i,j} is an element of a weight matrix W, and γ may be determined by ‘Cross-Validation’ and ‘Bootstrap’ methods, or by trial and error.

Early stopping: the training data set may be divided randomly into 2 subsets, wherein about 80% are used for training and about 20% are used for validation during training: After each epoch of training with the training subset, the network generalization performance is tested using the validation subset. The training is stopped after the error obtained utilizing the validation set is reduced beyond the tolerable deviation error, or at a local minimum of the validation-test error. Note that finding a local minimum is the common practice in NN training since practically it is impossible to find the global minimum. One has to scan the whole error surface which is huge. However, as long as the deviation error is tolerable, there is a small effect to whether it was found at a local or at a global minimum. To reduce this effect, a large number (e.g., NB) of different networks are trained for each Lead.

It should be noted that the final choice of the ‘model’, i.e., the NN architecture and dynamics, as well as improvement/optimization algorithms, may be determined by ‘Cross-Validation’ or ‘Bootstrap’ methods. These methods are aimed at estimating the mean generalization error (i.e., the mean squared error when testing the model with infinite number of new inputs) for each model:

$${E}_{generalization} = {lim}_{N \to ∞} {\sum }_{i = 1}^{N} \frac{{( {t}^{l} - {o}^{i} )}^{2}}{N}$$

Database segmentation: Heterogeneous database (i.e., including in the input matrix P ECGs of both male and female patients, in a wide range of ages, taking medications or not, smokers and non-smokers, etc.) may decrease the generalization performance of the NN. To solve the problem the database may be divided into more homogeneous subgroups, each containing patients of a single gender, from a small range of ages and similar in other parameters (smoking, medications). Each subgroup may be trained separately, yielding its own set of NNs. New patients will be diagnosed by the set of NNs matching their personal details (e.g., gender, age).

### The DHF Process

The Hidden Factor Diagnosis of the invention preferably combines NN classification with a unique signal processing and a test-set resampling process, which provide a reliable, ECG-based, diagnosis method.

### Generating the Training-Database

The steps of generating the training database are shown in the flowchart in FIG. 8. This process is initiated in step 80, in which standard rest-ECG signals are recorded from a large number of diagnosed, healthy and sick (CD of a given type, e.g., Ischemia, Cardiac Artery Disease—CAD), patients. In step 81 the recorded ECG signals are classified into separate homogeneous groups, for example, according to the following criteria: Gender, smoking, medication, age. Such a group may, for example, be defined to include only ischemic male patients, which are 40-50 years old, smoking and not taking any medications. Each of the groups is constructed to include N patients, preferably, half (N/2) of which are healthy, wherein the other half diagnosed with a CD (the number of patients N may vary from one group to another). The DHF process is preferably performed on each group separately, since in a homogeneous group, the main differentiating factor between healthy and sick patients is the CD factor (and not other factors such as gender or age).

Next, in step 82, the heart impulse signal data for each patient is acquired from selected ECG leads, preferably from leads 1, 5 and 12 (L_{I}, aVL and V6). It should be noted that the DHF process of the invention may be carried out utilizing other ECG lead signals, or with other type of heart activity signals, or with a combination thereof.

Moreover, the generation of the training database should also consider the following requirements:

1. The diagnosis of all patients must rely on catheterization or an equivalent trusted procedure;

2. The ECG signals should be digital, or transformed into a digital format;

3. The recording duration of the ECG signals should be around 10 seconds.

The following discussion refers to only one group of patients (e.g., Ischemic or with another CD), half of which (N/2) are diagnosed as healthy, and all others (N/2) patients are diagnosed as sick (diagnosed with CD) by a trusted procedure. The DHF of the invention is preferably carried out for each of the disease groups separately.

### Signal Processing

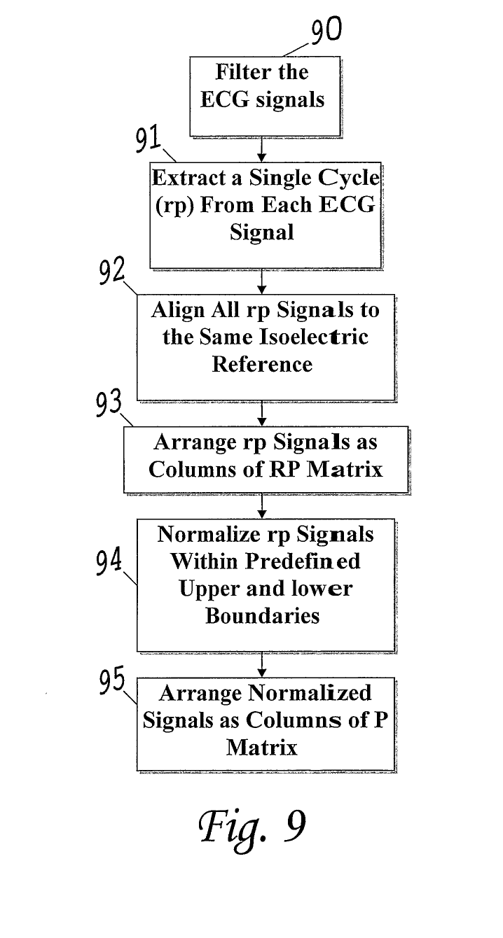

In a preferred embodiment of the invention the recorded ECG signals are at least 10 seconds long ECG recordings, preferably digitized with a sampling frequency rate of 500 Hz. Each ECG signal is preferably processed according to the processing steps shown in FIG. 9, which should be carried out on each of the selected ECG leads (e.g., 1, 5, and 12), of each of the N patients of the group.

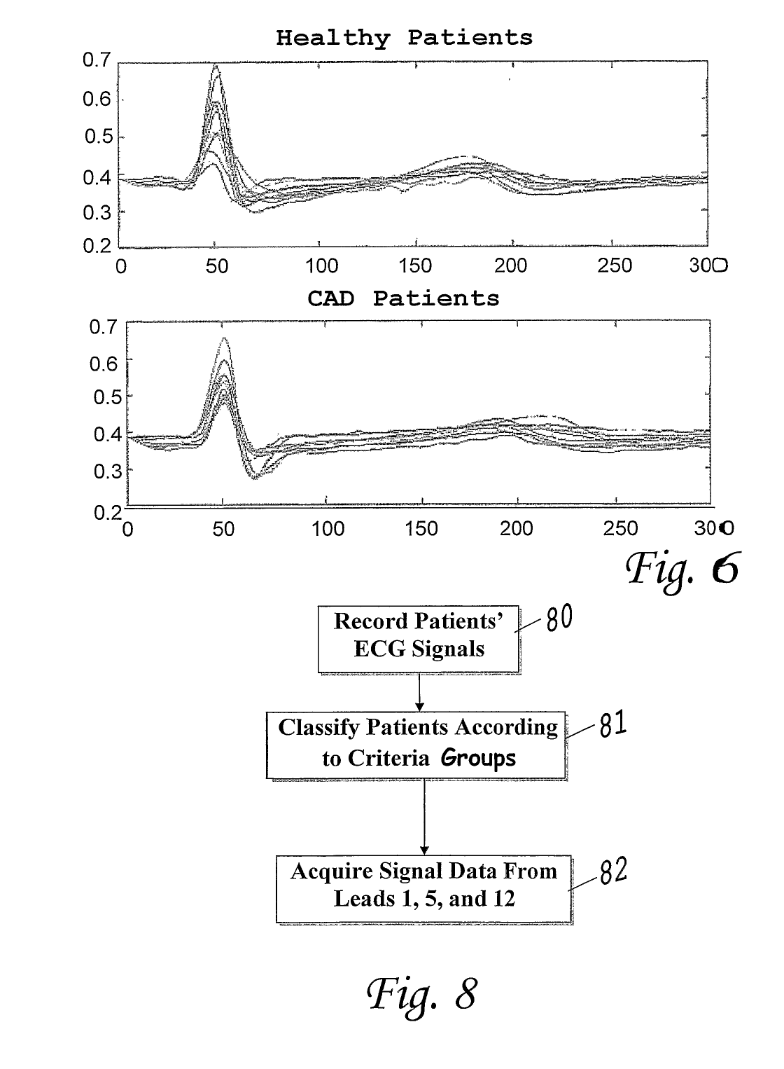

The processing starts in the filtering step 90, wherein the ECG signals are preferably filtered by a High-Pass Filter (e.g., a HPF with a cutoff frequency of 1 Hz, shown in FIG. 5B), a Notch filter (e.g., 50 and 150 Hz, shown in FIG. 5A) and a low-pass, EMG filter with a knee around 10 Hz shown in FIG. 5C. These filters are a common practice in ECG recordings.

In step 91, a raw-input signal, rpⁿ, is extracted from each sampled and filtered signal (for the n=1,2, . . . ,N patient). The raw-input signal is preferably a segment within a single heart cycle, as shown FIG. 4. The segment of the raw-input signal preferably starts 80 milliseconds to the left of the peak of the R-Wave (based on Lead 1), and is preferably 600 milliseconds long. If a sampling rate of 500 Hz is used for digitizing the ECG signal, the raw-input signal rpⁿ obtained comprises 300 samples, i.e., a column vector of 300 elements. As will be described herein below, the rpⁿ signals of all patients are centered horizontally about a common point, which is preferably the peak of the R-Wave along the time axis (as shown in FIG. 6).

In step 92, the rpⁿ signals of each lead of all of the N patients are aligned in order to obtain a common isoelectric reference value, i.e., the raw-input vectors are shifted “up” or “down” so that the 1^{st} element in the rpⁿ vectors has the same value for all n signals, as demonstrated in FIG. 6. After aligning the signals, in step 92, all the raw-input vectors are packed in a d×N RP matrix, such that each column in the RP matrix is a raw-input vector rpⁿ.

Next, in step 94, the raw-input vectors rpⁿ in the RP matrix are normalized within predetermined upper and lower boundaries (preferably within the range [0.25, 0.75]), thus maintaining relative amplitudes. This normalization step may be carried out by computing

$${p}^{n} = 0.5 \frac{{rp}^{n} - min ( RP )}{max ( RP ) - min ( RP )} - 0.25$$

for each raw-input vector rpⁿ, where max(RP) and min(RP) are the largest and smallest elements in the raw-input matrix RP, respectively. Finally, in step 95, the normalized vectors pⁿ are packed in a d×N input matrix P. The columns of the input matrix P are preferably arranged in 2 subgroups, as follows: columns 1 to N/2 are preferably populated with the input vectors of the healthy patients, and columns N/2+1 to N with the input vectors of the CD (sick) patients.

After carrying out the above steps, the input matrix P is obtained, comprising normalized ECG signals pⁿ, as illustrated in FIG. 6.

### Generating the DHF Diagnosing Set

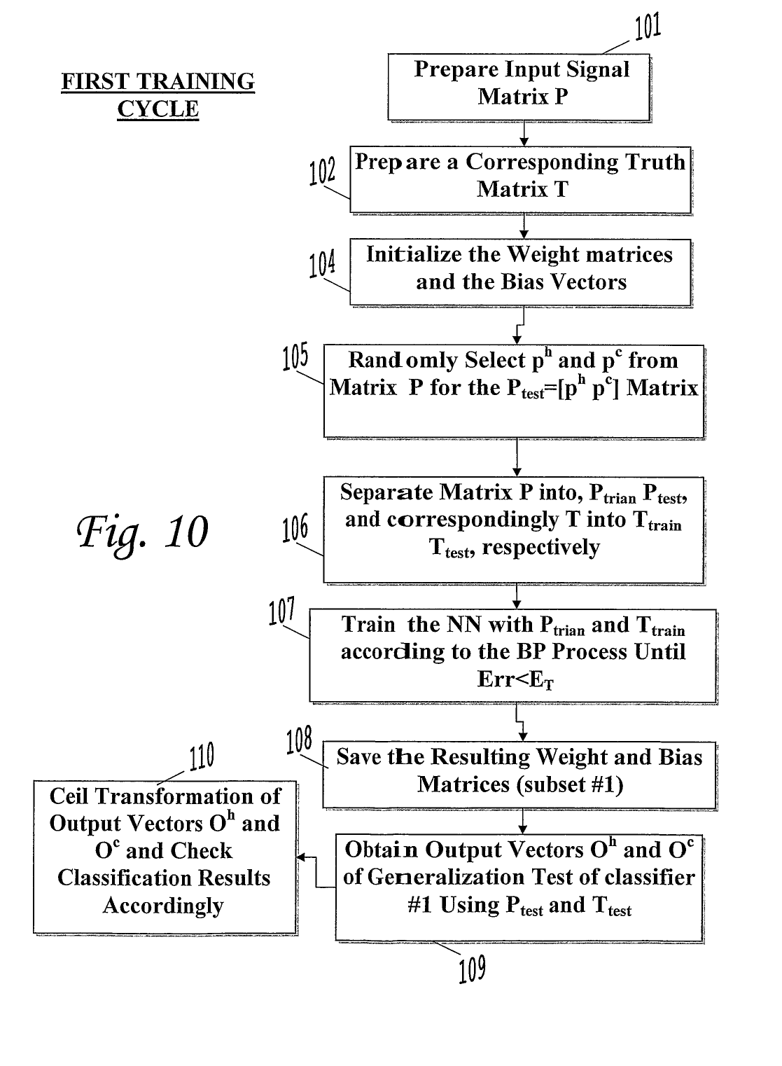

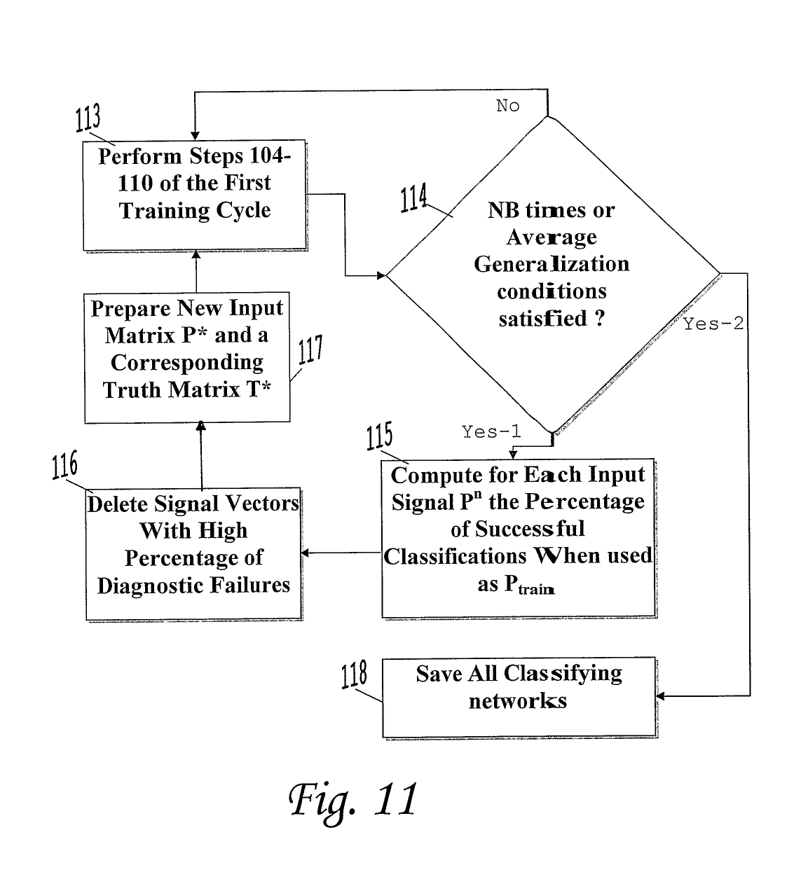

In order to diagnose new patients, based on their rest-ECG recording, the DHF process of the invention employs a large set of matrices and vectors that will be referred to as the complete diagnosing set. The complete diagnosing set contains a large number of NNs, represented by real-valued weight matrices and bias vectors. These matrices and vectors are obtained through the process of NN training and test-set resampling which is discussed in details hereinafter with reference to FIGS. 10 and 11.

### The 1st Training Cycle

For the sake of simplicity, in the following discussion the training of a 3-layered (input, intermediate and output) NN is exemplified. Obviously, this example does not limit the NN of the invention, which may comprise any different number of intermediate layers. It should be noted that the NN of the invention is preferably implemented utilizing the logistic transfer function.

FIG. 10 is a flow chart illustrating a preferred process for carrying out the first training cycle for a given ECG Lead (e.g., Lead 1, 5 or 12). The process is started in step 101 wherein a d×N input matrix P is constructed as was explained in details herein above with reference to FIG. 9 (Each column of the P matrix is a processed signal of Lead 1, of a given patient). The corresponding 2×N ‘truth’ matrix T is constructed in step 102, such that each 2-element column in T is either

$${t}^{n} = [ \begin{matrix}1 \\ 0\end{matrix} ]$$

(for healthy patient, 1≦n≦N/2) or

$${t}^{n} = [ \begin{matrix}0 \\ 1\end{matrix} ]$$

(for a CD patient, N/2<n≦N). The weights matrices, W¹ and W², and the bias vectors, b¹ and b², are initialized in step 104 with small random values, as was described herein before.

In step 105, two columns of the input matrix P are randomly selected, where one belongs to a healthy patient—p^{h} (1≦h≦N/2), and the other belongs to a CD patient—p^{c} (N/2<c≦N). The P and T matrices are separated in step 106 into a ‘train’ and ‘test’ sub-matrices, wherein Pₜᵣₐᵢₙ and Tₜᵣₐᵢₙ are the sub-matrices of P and T, respectively, in which the h and c columns are omitted, i.e., Pₜᵣₐᵢₙ=└p¹, . . . , p^{h-1}, p^{h+1}, . . . , p^{N/2}, p^{N/2+1}, . . . , p^{c-1}, p^{c+1}, . . . , P^{N}┘ and Tₜᵣₐᵢₙ=└t¹, . . . ,t^{h-1},t^{h+1}, . . . ,t^{N/2},t^{N/2+1}, . . . ,t^{c-1},t^{c+1}, . . . ,t^{N}┘, while the Pₜₑₛₜ=[p^{h}, p^{c}] and Tₜₑₛₜ=[t^{h},t^{c}] are composed of the h and c columns. Namely, Pₜᵣₐᵢₙ is a d×(N−2) matrix, Tₜᵣₐᵢₙ is a 2×(N−2) matrix, Pₜₑₛₜ is a d×2 matrix [p^{h}, p^{c}], and Tₜₑₛₜ is a 2×2 matrix

$$[ \begin{matrix}1 & 0 \\ 0 & 1\end{matrix} ] .$$

In step 107, the network is trained using the Pₜᵣₐᵢₙ and Tₜᵣₐᵢₙ matrices according to the BP algorithm which was previously described hereinabove. When the network reaches the desired performance, i.e., Err=Σ_{all-signals}[Tⁿ−V^{M}]²=E_{T}, the corresponding weight matrices and bias vectors resulting from this training process e.g.: W₁¹, W₁², b₁¹, b₁², are saved in step 108. These results comprise the first classifier, referred to herein as the classifying network #1. In step 109, the classifying network #1 is tested for generalization, using the Pₜₑₛₜ and Tₜₑₛₜ matrices, namely:

O^{#1}=ƒ(W₁²·ƒ(W₁¹·Pₜₑₛₜ+b₁¹)+b₁²)
where f is the transfer (preferably the logistic) function.

Next, in step 110, The elements oᵢⱼ of the resulting output

$${O}^{\#1} = [ {O}^{h} {O}^{c} ] = [ \begin{matrix}{o}_{11} & {o}_{12} \\ {o}_{21} & {o}_{22}\end{matrix} ]$$

are transformed into 0 and 1 values using the ceil function, as follows: oᵢⱼ=ceil(oᵢⱼ−0.5), where ‘ceil’ rounds its operands to the nearest largest integer (e.g., ceil(−0.3)=0, ceil(0.1)=1). This may yield one of the following four possible results, that are compared with Tₜₑₛₜ:

i)

$${O}^{\#1} = [ \begin{matrix}1 & 0 \\ 0 & 1\end{matrix} ]$$

i.e., classifying network #1 has correctly classified both healthy and sick test patients—100% success;

ii)

$${O}^{\#1} = [ \begin{matrix}1 & 1 \\ 0 & 0\end{matrix} ]$$

i.e., classifying network #1 has correctly classified only the healthy patient—50% success;

iii)

$${O}^{\#1} = [ \begin{matrix}0 & 0 \\ 1 & 1\end{matrix} ]$$

i.e., classifying network #1 has correctly classified only the sick patient—50% success;

iv)

$${O}^{\#1} = [ \begin{matrix}0 & 1 \\ 1 & 0\end{matrix} ]$$

i.e., classifying network #1 has failed to classify both test patients—0% success.

### Test-Set Resampling Process

The test-set resampling steps are illustrated in the flowchart of FIG. 11. These steps are performed further to the basic training steps which were described above, in order to initiate the DHF of the invention. In steps 113 and 114, the first training cycle steps 104 through 110, described above with reference to FIG. 10, are preferably repeated NB times

$$( e . g . , NB \approx 0.5 {( \frac{N}{2} )}^{2} ) ,$$

or until the average generalization performance of all training cycles reaches an asymptotic value (Yes-1, i.e., the first time one of the conditions in step 114 is satisfied). This will result in NB classifying networks that constitute a temporary diagnosing-set, e.g., for the three layered NN exemplified above the NB temporary classifying networks are:

$$\{ \begin{matrix}{W}_{1}^{1} & {W}_{1}^{2} & {b}_{1}^{1} & {b}_{1}^{2} \\ ⋮ & ⋮ & ⋮ & ⋮ \\ ⋮ & ⋮ & ⋮ & ⋮ \\ {W}_{NB}^{1} & {W}_{NB}^{2} & {b}_{NB}^{1} & {b}_{NB}^{2}\end{matrix} \}$$

wherein each row in the above array represents one classifying NN.

In a preferred embodiment of the invention, the average generalization performance is determined utilizing a grading scheme. For each cycle (namely, each classifying network) 3 success ‘grades’ are determined: i) one grade for success in diagnosing the healthy signal (0 or 100%); ii) second grade for success in diagnosing the sick signal (0 or 100%); and iii) a general grade (both healthy and sick—0, 50 or 100%), where the average generalization performance values are actually the averages of these three grades over the currently tested classifying networks.

At this point, each input pⁿ is examined: The ceil function transformation described above is used to compute for each input signal vector pⁿ, the percentage of classifying networks that were successful in classifying pⁿ, when it was used as a training input in Pₜᵣₐᵢₙ. In steps 115 and 116, the pⁿ vectors for which a certain percentage (e.g., 60%) of the classifying networks failed to classify during the training process are deleted. Subsequently, in step 117, a new input matrix P* is obtained, wherein the number of inputs vectors pⁿ is reduced, such that the dimensions of the inputs matrix P* obtained now are d×N*, where N*<N. Correspondingly, the matching 2×N* ‘truth’ output matrix T* is constructed by eliminating the corresponding truth vectors according to the deleted input vectors.

The training steps performed in steps 113 and 114, are repeated while utilizing the modified matrices P* and T*, and once completed (Yes-2, i.e., the second time one of the conditions in step 114 is satisfied), in step 118, the final classifying networks obtained are saved, yielding the final diagnosing set of the trained Lead. e.g.:

$$\{ \begin{matrix}{W}_{1}^{{ }^{*} 1} & {W}_{1}^{{ }^{*} 2} & {b}_{1}^{{ }^{*} 1} & {b}_{1}^{{ }^{*} 2} \\ ⋮ & ⋮ & ⋮ & ⋮ \\ ⋮ & ⋮ & ⋮ & ⋮ \\ {W}_{NB}^{{ }^{*} 1} & {W}_{NB}^{{ }^{*} 2} & {b}_{NB}^{{ }^{*} 1} & {b}_{NB}^{{ }^{*} 2}\end{matrix} \}$$

This process (illustrated in FIGS. 10 and 11) is carried out for each ECG Lead signal used by the DHF, preferably Leads: 1, 5 and 12, such that the complete diagnosing set is composed of 3 such final diagnosing sets. This complete set is employed in classifying new, unfamiliar ECG signals.

### Classifying a New ECG Signal

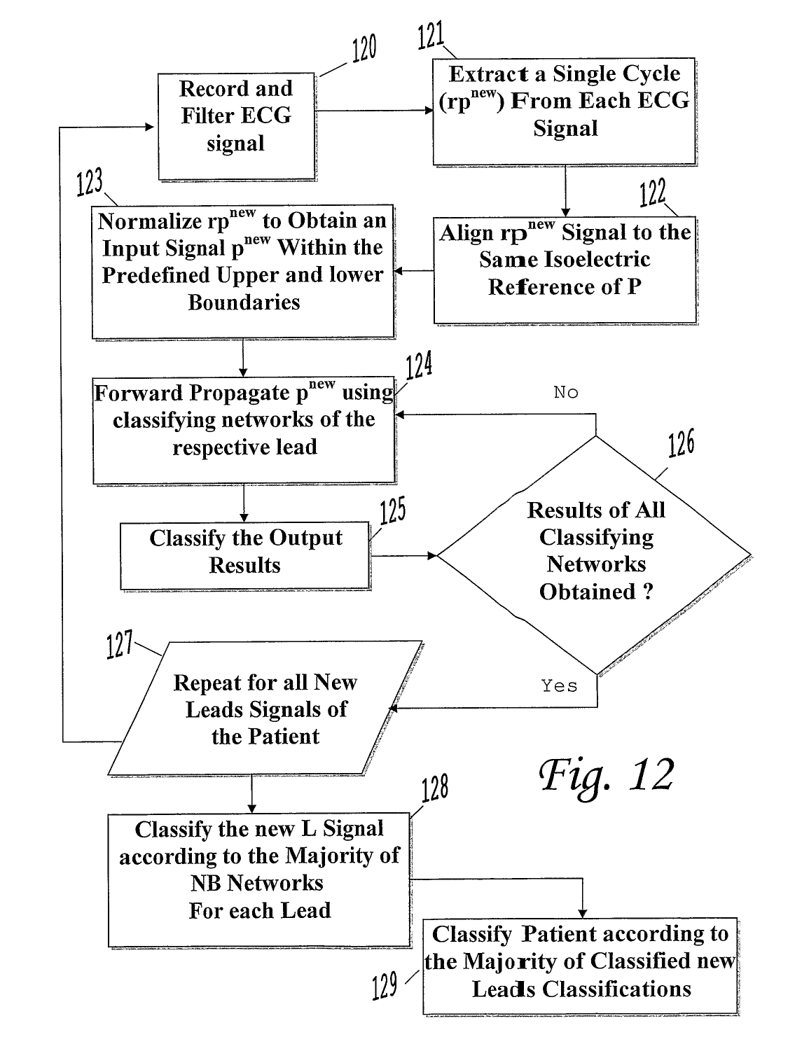

Classifying ECG signals of non-diagnosed patients by the DHF can now be carried out as illustrated in the flowchart of FIG. 12. For this purpose, the digital rest-ECG signals of a patient are recorded and maintained, preferably utilizing the signals obtained form leads 1, 5, and 12. The process starts in step 120, wherein the ECG signal obtained from Lead 1 is filtered, preferably via the High-Pass, notch, and EMG filters which were previously described. In the following step, step 121, a cycle segment is extracted from the filtered Lead 1 signal as was previously described with reference to FIG. 4, in order to obtain a new raw-input signal (column vector) rp^{new}.

In step 122, the new raw-input signal rp^{new} is aligned to the same isoelectric reference value that was employed in preparing the input signal matrix P. In the normalization step 123, normalization of the new raw-input signal rp^{new} is carried out within the same bounds as used in the preprocessing steps of the input vector pⁿ of P, namely:

$${p}^{new} = 0.5 \frac{ {rp}^{new} - min ( RP )}{max ( RP ) - min ( RP )} - 0.25 .$$

Then in step 124 the new input signal p^{new} is forward propagated through the classifying network #1 (of Lead 1), e.g., in the three-layers NN example:

O=ƒ²(W₁²·ƒ¹(W₁¹·p^{new}+b₁¹)+b₁²)

which results in an output vector

$$O = [ \begin{matrix}{o}_{1} \\ {o}_{2}\end{matrix} ] .$$

In step 125 the signal is classified as ‘healthy’ if o₁>0.5 and o₂<0.5, or as ‘CD’ if o₁<0.5 and o₂>0.5. In step 126, it is checked if the new signal was classified using all the classifying networks of Lead-1, and it returns the control to step 124 until classification of the new signal is carried out with all NB classifying networks (or less if generalization average had been reached asymptotic value in the training step). Step 127 returns the control to steps 120 in order to repeat the classification process of steps 120 through 126 for the remaining ECG signals, e.g., of leads 5, 12.

The final classification is based on majority decision rules, performed in two step: First, in the classification of step 128, each of the ECG signals, of each lead, is classified independently as healthy or sick according to the classification of the majority (e.g., >50%) of the NB classifying networks of the given lead. For example, if p^{new} of Lead 1 is classified as ‘healthy’ by more than NB/2 classifying networks, it will be classified as ‘healthy’ for that Lead.

Finally, in step 129, the signal is diagnosed according to the classifications of the majority of the leads, e.g., if the signal was classified as CD by the classification process performed with at least two of the three leads, it will be diagnosed as CD.

### System Overview

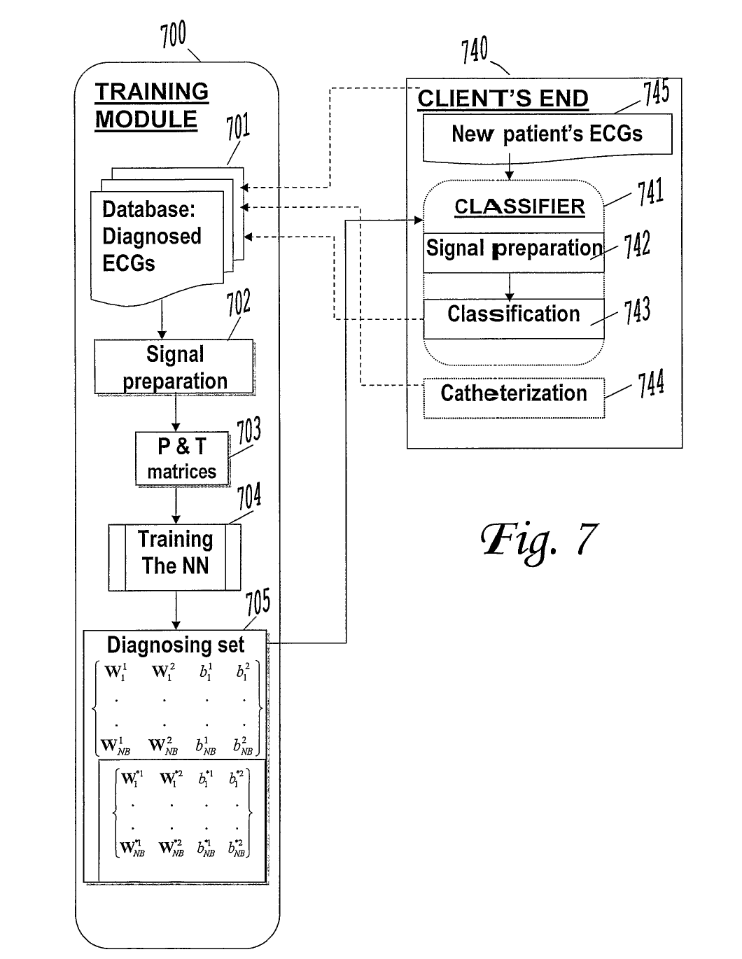

FIG. 7 is a block diagram illustrating a system, capable of carrying out the DHF of the invention. The system preferably comprises two main modules, a Training Module 700, for instance, a computer program operating on a central server, and the Classifier Module 740 (Client's End), for instance, a computer program operating on the PC, Palm, or a dedicated diagnosing device of the client (physician, patient).

The Training Module 700 operates in the background. It scans the current database 701 of diagnosed ECGs and produces the updated complete diagnosing-set matrices and vectors. Each new ECG diagnosed signal that is added to the database is processed by a signal preparation module 702, which updates the P and T matrices maintained in 703 (e.g., the server memory). The diagnosing-set is updated by the training module 704 and maintained in 705 (e.g., server memory).

The complete diagnosing-set is installed on the classifier 741, and is updated periodically (see arrow from 705 to 741). Whenever a new ECG signal 745, of a non-diagnosed patient, is obtained by the classifier 740, it is preprocessed by the signal preparation module 742 (which is identical to module 702), and classified by the classification module 743, according to the DHF of the invention.

The training module 700 stores a large set of ECG recordings 701, which are diagnosed a-priori by expert cardiologists, based on catheterization or equivalent procedure. For each cardiac disease there exists a separate database and a matching complete diagnosing set. The ECG databases are constructed such that about half of the patients are diagnosed as healthy, and the rest are sick.

It should be noted that in a preferred embodiment of the invention, all ECG signals (healthy and sick) of the database are visually diagnosed as healthy, namely, the standard rule-based and visual diagnostic methods do not apply for these ECGs. In this way it is assured that tile factors extracted by the training process are the relevant hidden factors of the cardiac disease. Similarly, a mirror database should be employed, wherein all ECG signals (healthy and sick) are visually diagnosed as sick. Combining diagnosis from both databases will reduce ‘false negative’ and ‘false positive’ errors.

The databases of ECG signals are processed by the signal preparation modules according to the processing steps described with reference to FIG. 9. The training process shown in FIGS. 10-11 is carried out by the training module 704, and the resulting complete diagnosing-sets, are saved, preferably on a CDROM, and/or transferred to the client via the internet, or other data storing media or data communication means.

The ECG signal 745 of a new non-diagnosed patient is digitally recorded and provided to the classifier 741 that classifies the signal 745 according to the DHF classification process of the invention. If the patient is further diagnosed by catheterization (744), the ECG signals and the diagnostic results are added to the database of the training module for increasing it and improving the diagnosing-set (dashed arrows in FIG. 7).

The above examples and description have of course been provided only for the purpose of illustration, and are not intended to limit the invention in any way. As will be appreciated by the skilled person, the invention can be carried out in a great variety of ways, such as processing rest ECG, stress-test ECG or Holter-test ECG signals, employing techniques different from those described above, all without exceeding the scope of the invention.

1. A method for diagnosing silent and/or symptomatic cardiac diseases in human patients, based on extracting and analyzing hidden factors or a combination of hidden and known factors of ECG signals, comprising:
a) acquiring raw, pre-processed ECG signals of a group of diagnosed patients, some of which are a-priori diagnosed as sick patients while the remaining patients are a-priori diagnosed as healthy patients by a trusted procedure, wherein both the healthy and the sick patients were diagnosed as being all healthy, according to standard, rule-based, visual methods of ECG diagnosis;
b) iteratively training artificial neural networks to accurately classify said diagnosed patients, while excluding the ECG signals of one or more patients thereby constituting a test-set, by means of pattern-recognition, preformed by processing their corresponding raw input signals, each input signal comprising essentially a single heart cycle, while whenever required, adding trained network iterations, until predetermined training performance conditions are satisfied;
c) saving the neural network's weights and biases representing the hidden factors which discriminate the ECG signal patterns of healthy and sick patients from one another; and
d) diagnosing unknown patients from said test-set, as well as new patients that were not included in the selected diagnosed group as sick or healthy patients by processing their corresponding raw signals based on the hidden factors represented by said trained neural networks.

2. A method according to claim 1, wherein steps (b)-(d) are repeated NB times using different test-sets in each repetition, until the average generalization performance of the test-sets of all NB training cycles reaches an asymptotic value.

3. A method according to claim 1, comprising:
a) acquiring rest-ECG signals of diagnosed patients, some of which are a-priori diagnosed as sick patients and the remaining patients are a-priori diagnosed as healthy patients by trusted procedures, wherein both the healthy and the sick patients were diagnosed as being all healthy or as being all sick, according to standard, rule-based, visual methods of ECG diagnosis;
b) processing said raw signals to obtain filtered input-signals, each defined within a single heart cycle, aligned about the same isoelectric reference and normalized within predefined boundaries;
c) randomly separating signals of sick and healthy patients into ‘train’ and ‘test’ sets, where each set comprises signals of both ‘healthy’ and ‘sick’ patients;
d) iteratively training a Feed Forward artificial neural network to correctly classify said diagnosed patients, by forwarding the signals of the train-set through the network, comparing the network output with the trusted diagnosis, and updating weights and biases data of the network accordingly, where each time, inputs that correspond to the diagnosed patients are fed into the network, while providing weights and biases data to each cycle, and updating these weights and biases according to error minimization techniques, until a predetermined training performance condition is satisfied or deteriorated;
e) testing the trained network by processing the inputs that correspond to the selected test-set signals by the network and maintaining the test results of said trained network.
f) adding trained networks by repeating steps c) to e) above NB times, until a predetermined test-performance condition, based on the aggregated test results of all trained networks, is satisfied;
g) disqualifying inputs that consistently contributed a significant error in the training process of the trained networks.
h) deleting all trained networks and repeating the training process of steps c) to f) with the reduced set of inputs;
i) repeating the above process for a number of ECG Lead signals;
j) saving the final weights and biases data obtained by the training of each of said neural networks;
k) acquiring new rest-ECG signals of unknown patients that were not included in the training phase;
l) processing said new signals to obtain new filtered input-signals aligned about the same isoelectric reference and normalized using the same formula that was applied for processing the a-priori diagnosed signals;
m) applying said new signals to inputs of said trained neural networks while utilizing the saved weights and biases data, and transforming the output results of each new signal to obtain a “sick” or “healthy” classification;
n) classifying each of said new signals as sick or healthy according to the majority of the classifications results obtained by all NB trained neural networks for each said signal, for each lead separately; and
o) diagnosing each of said unknown patients according to the majority of Leads classifications of said new signals, while considering the majority of results obtained from the various ECG Leads.

4. A method according to claim 3, wherein processing of the raw signal is performed by the following steps:
a) filtering each acquired signal;
b) extracting a raw-input signal from each of said filtered signals, wherein said raw-input signal comprises a segment within a single heart cycle;
c) aligning said raw-input signals about the same isoelectric reference; and
d) normalizing said aligned raw-input signals within predetermined upper and lower boundaries.

5. A method according to claim 3, wherein diagnosis of new patients (i.e., generalization) is optimized by any combination of generalization-improvement techniques: Optimizing the NN architecture and/or ‘regularization’ of the performance function and/or ‘early stopping’ of the training process and/or employing an optimized training process.

6. A method according to claim 4, wherein the single cycles extracted from each of the signals are of the same time interval, and taken starting at the same predefined time interval before the peak of the R-wave of that cycle.

7. A method according to claim 4, wherein the single cycle time interval is about 600 milliseconds.

8. A method according to claim 4, wherein the predefined time interval is about 80 milliseconds.

9. A method according to claim 4, wherein the upper bound is larger than 0.75 and smaller than 1 and the lower bound is smaller than 0.25 and larger than 0.

10. A method according to claim 3 wherein whenever required, the processing step comprises converting the ECG signals into digital format.

11. A method according to claim 1, wherein the trusted procedure is catheterization.

12. A method according to claim 1, wherein the ECG signals are rest ECG, and/or stress-test ECG.

13. A method according to claim 1, wherein training is performed using error minimization and/or error back propagation techniques.

14. A System for diagnosing silent and/or symptomatic cardiac diseases in unknown human patients, based on extracting and analyzing hidden factors or a combination of hidden and known factors of ECG signals, comprising:
a) a database of a-priori diagnosed ECG signals of sick and of healthy patients, wherein the diagnosis of said patients was obtained a-priori via trusted procedures and wherein both the healthy and the sick patients were diagnosed as being all healthy or as being all sick, according to standard, rule-based, visual methods of ECG diagnosis;
b) at least one signal processing unit for digitizing and processing said signals and for iteratively training artificial neural networks to accurately classify said diagnosed patients by processing their corresponding raw input data while whenever required, adding trained network cycles, until a predetermined training performance condition is satisfied;
c) a memory for saving the weights and biases data representing the trained neural networks; and
d) a classification module for diagnosing unknown patients as sick or healthy patients by processing their corresponding raw signals by said trained neural networks.

15. A system according to claim 14, comprising:
a) a database of diagnosed ECG signals of sick and of healthy patients, a-priori diagnosed as sick patients and of patients a-priori diagnosed as healthy patients by trusted procedures wherein both the healthy and the sick patients were diagnosed as being all healthy or as being all sick, according to standard, rule-based, visual methods of ECG diagnosis;
b) at least one signal processing unit for digitizing and processing said signals so as to obtain filtered input-signals aligned about the same isoelectric reference by shifting the raw input vectors rpⁿ, before normalization, so that the first element in each rpⁿ vector has the same value for all n signals and normalized within predefined boundaries so as to produce normalized pⁿ vectors and for producing and utilizing weights and biases data obtained via a training process of artificial neural networks;
c) a memory for saving weights and biases data of artificial neural networks; and
d) a classification module for acquiring new ECG signals of a non-diagnosed patient, and processing said new signals to obtain new filtered input-signals aligned about the same isoelectric reference and normalized within the same predefined boundaries used by said signal processing unit, said classification module comprises sets of artificial neural networks for diagnosing said new signals utilizing the weights and biases data stored in said memory.

16. A system according to claim 15, further comprising a training unit for training and testing the training of artificial neural networks, in which
a) the training is performed by randomly selecting signals of sick and healthy patients from the database of a-priori diagnosed ECG signals and is continuously carried out until predetermined training and generalization performance conditions are satisfied; and
b) step (a) is repeated NB times until the average generalization performance of all NB training cycles reaches an asymptotic value.

17. A system according to claim 16, wherein
a) the training is performed by the training unit whenever a new a-priori diagnosed ECG signal is added to the database;
b) the new weights and biases data obtained are stored in the memory and used for the diagnosis performed by the classification unit; and
c) steps (a) and (b) are repeated NB times until the average generalization performance of all NB training cycles reaches an asymptotic value.

18. A system according to claim 15, wherein the processing unit includes:
a) filters for removing interfering signals from the cardiac signal; and
b) processing means for extracting a raw-input signal from the filtered signals, wherein said raw-input signal comprises a segment within a single cycle, and for aligning said raw-input signals about the same isoelectric reference; and for normalizing said aligned raw-input signals within predetermined upper and lower boundaries.

19. A system according to claim 18, wherein the single cycles extracted from each of the signals are of the same time interval, and taken starting at a predefined time interval before the peak of a R-wave.

20. A system according to claim 19, wherein the single cycle time interval is about 600 milliseconds.

21. A system according to claim 19, wherein the predefined time interval is about 80 milliseconds.

22. A system according to claim 18, wherein the upper bound is between 0.75 and 1 and the lower bound is between 0 and 0.25.

23. The method according to claim 1 wherein the artificial neural networks are trained using preprocessed ECG signals of a group of diagnosed patients that includes patients that are diagnosed as being sick and other patients that are diagnosed as being healthy according to both standard, rule based, visual methods of ECG diagnosis and trusted procedures

24. The system according to claim 14 wherein the artificial neural networks are trained using preprocessed ECG signals of a group of patients that includes healthy patients that are diagnosed as being healthy and the sick patients are diagnosed as being sick according to both standard, rule based, visual methods of ECG diagnosis and trusted procedures.

25. A system according to claim 24, wherein the single cycle time interval is about 600 milliseconds.

26. A system according to claim 24, wherein the predefined time interval is about 80 milliseconds.

27. A system according to claim 22, wherein the upper bound is between 0.75 and 1 and the lower bound is between 0 and 0.25.

28. A system according to claim 18, wherein the digitizing is carried out utilizing a sampling frequency of about 500 Hz.

29. A system according to claim 18, wherein the training is carried out utilizing signals of healthy and sick patients which are all visually diagnosed as healthy.

30. A method according to claim 18, wherein the training is carried out utilizing signals of healthy and sick patients which are all visually diagnosed as sick.
